(19) United States
(12) Patent Application Publication
(10) Pub. No.: US 2022/0108798 A1
(43) Pub. Date: Apr. 7, 2022

(54) SYSTEM, METHOD, AND COMPUTER PROGRAM FOR SAFELY MANAGING INTERACTIONS AMONG INDIVIDUALS

(71) Applicant: Kevin J. Zilka, Los Gatos, CA (US)

(72) Inventor: Kevin J. Zilka, Los Gatos, CA (US)

(21) Appl. No.: 17/341,167

(22) Filed: Jun. 7, 2021

Related U.S. Application Data
(60) Provisional application No. 63/087,071, filed on Oct. 2, 2020.

(51) Int. Cl. G16H 40/67; G16H 40/20; G06Q 10/10

(52) U.S. Cl. CPC G16H 40/67; G06Q 10/1095; G16H 40/20

(57) ABSTRACT

A system, method, and computer program are provided for: receiving first information regarding a virus infection status of a first person; receiving second information regarding a virus infection status of a second person; based on the first information and the second information, conditionally scheduling a meeting between the first person and the second person.

### CROSS-REFERENCE TO RELATED APPLICATIONS

This application claims the benefit of U.S. Provisional Application No. 63/087,071 (Attorney Docket No. MARC1P002+) titled “SYSTEM, METHOD, AND COMPUTER PROGRAM FOR SAFELY MANAGING INTERACTIONS AMONG INDIVIDUALS,” filed Oct. 2, 2020, the entire contents of which is incorporated herein by reference.

### FIELD OF THE INVENTION

The present invention relates to animal and human safety.

### BACKGROUND

Pandemics are now a regular fact-of-life for twenty-first century mankind. For example, COVID-19 has significantly impacted the nature and amount of interactions among humans. Such interactions now pose a threat to safety with respect to the transmission of viruses.

As such, there is thus a need for addressing these and/or other issues associated with the prior art.

### SUMMARY

A system, method, and computer program are provided for: receiving first information regarding a virus infection status of a first person; receiving second information regarding a virus infection status of a second person; based on the first information and the second information, conditionally scheduling a meeting between the first person and the second person.

### BRIEF DESCRIPTION OF THE DRAWINGS

FIG. 1 illustrates a method for managing an interaction among individuals, in accordance with one embodiment.

FIG. 2 illustrates a flow chart of managing an interaction among individuals in connection with pet grooming appointment, in accordance with one embodiment.

FIGS. 3A-B illustrate a variety of user interfaces associated with various operations outlined in FIG. 2, in accordance with one embodiment.

FIG. 4 illustrates an exemplary mobile pet grooming facility with a variety of disinfecting systems.

FIG. 5 illustrates a network architecture, in accordance with one possible embodiment.

FIG. 6 illustrates an exemplary system, in accordance with one embodiment.

### DETAILED DESCRIPTION

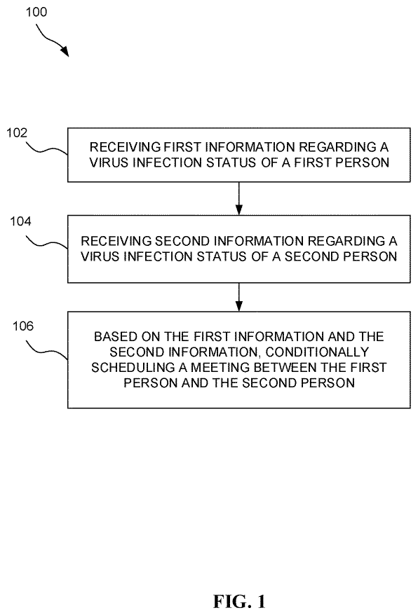

FIG. 1 illustrates a method 100 for managing an interaction among individuals, in accordance with one embodiment.

As shown, first information regarding a virus infection status of a first person is received. See operation 102. In one embodiment, first information may include predetermined dates and times when the first person is known to be available for a meeting. In another embodiment, first information may include a pre-defined address from which a proximity algorithm may determine available meeting locations. The first information may also include the current location of the first person from which a proximity algorithm may determine proposed meeting locations. In yet another embodiment, first information may include virus infection status information in the form of a date of a negative test result. In still another embodiment, first information may include a date of a positive test followed by quarantine and remedial measures.

Additionally, second information regarding a virus infection status of a second person is received. See operation 104. In one embodiment, second information may include predetermined dates and times when the second person is known to be available for a meeting. In another embodiment, second information may include a pre-defined address from which a proximity algorithm may determine available meeting locations. The second information may also include the current location of the second person from which a proximity algorithm may determine proposed meeting locations. In yet another embodiment, second information may include virus infection status information in the form of a date of a negative test result. In still another embodiment, second information may include a date of a positive test followed by quarantine and remedial measures.

Further, a meeting is conditionally scheduled between the first person and the second person based on the first information and the second information. See operation 106. In one embodiment, the conditionally scheduled meeting may include a proposed time of meeting. In another embodiment, the conditionally scheduled meeting may include a meeting duration. In yet another embodiment, the conditionally scheduled meeting may include a proposed location.

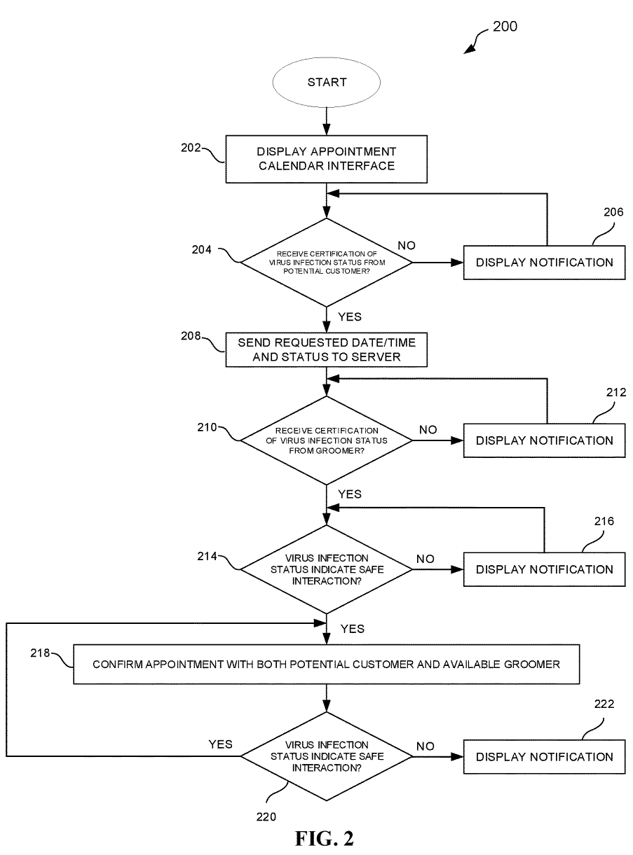

FIG. 2 illustrates a flow chart 200 of managing an interaction among individuals in connection with pet grooming appointment, in accordance with one embodiment. As an option, the flow chart 200 may be implemented in the context of any one or more of the embodiments set forth in any previous and/or subsequent figure(s) and/or description thereof. Of course, however, the flow chart 200 may be implemented in the context of any desired environment. Further, the aforementioned definitions may equally apply to the description below.

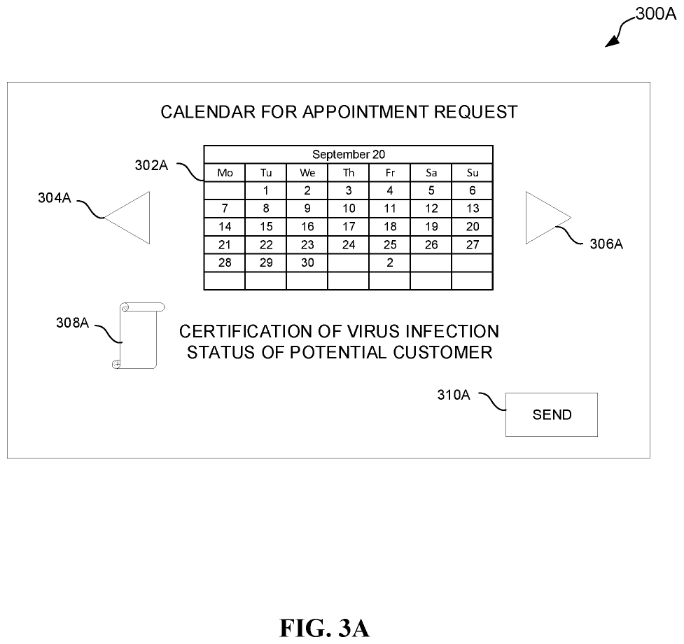

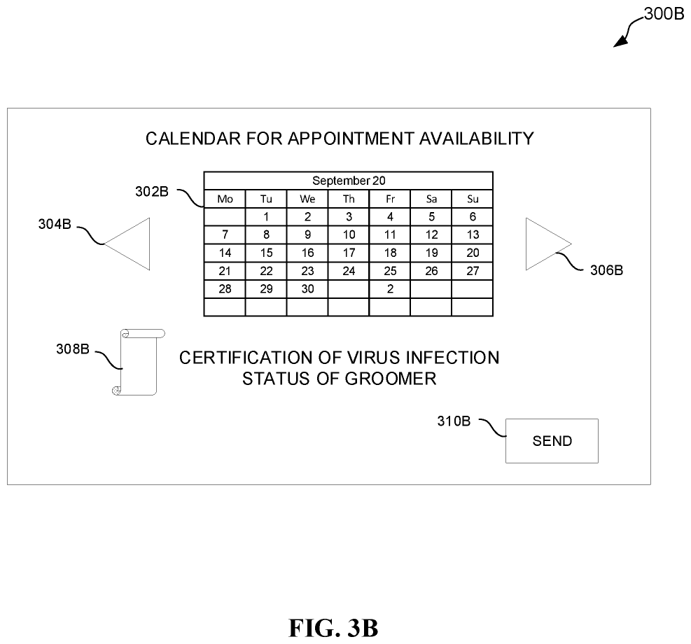

In operation, a potential customer is presented with an appointment counter interface 202 wherein the potential customer may display and specify a variety of information. See also FIG. 3B for details.

Additionally, a potential customer may receive certification of virus infection status of said potential customer. See operation 204. If the potential customer receives a valid certification, then the potential customer may choose to “Send” status and request a date and time for a scheduled meeting to a server. See event 208. If the potential customer does not receive a valid certification, the interface may display a notification 206 to the potential customer and may cycle back to a state of awaiting certification before the potential customer may send a meeting request to the server.

In addition, a groomer may receive certification of virus infection status of said groomer. See operation 210. If the groomer receives a valid certification, then the groomer may choose to “Send” date and time availability for a scheduled meeting to a server. If the potential customer does not receive a valid certification, the interface may display a notification 212 to the groomer and may cycle back to a state of awaiting certification before the groomer may send availability to the server.

Further, said server, having received certification and a meeting request from a potential customer as well as certification and availability information from the groomer, may determine whether the known virus infection status indicates a safe interaction. See operation 214. If said server determines that the virus infection status indicates a safe interaction, then the server may confirm an appointment between the potential customer and available groomer. See event 218. If said server does not determine that virus infection status indicates a safe interaction, the interface may display a notification 216 and may cycle back to a state of awaiting virus infection status indicating a safe interaction before confirming an appointment.

Further still, after confirmation of an appointment between the potential customer and available groomer 218, said server may again verify whether infection status indicates a safe interaction. See operation 220. If said server determines that the virus infection status indicates a safe interaction, then the server may maintain the “confirmed” categorization of the appointment between the potential customer and available groomer. See event 218. If, upon new or changed information, said server does not determine that virus infection status indicates a safe interaction, the interface may display a notification 222 and unconfirm the appointment.

FIG. 3A illustrates a potential customer's user interface associated with operations outlined in FIG. 2, in accordance with one embodiment. As an option, the potential customer's user interface may include a visual calendar 302A designed to allow selection of a potential day for a scheduled meeting. In addition, the potential customer's user interface may include an advance button 306A and a back button 304A designed to move between subsequent and previous calendar months. Further, the potential customer's user interface may include indication of the potential customer's virus infection status certification 308A. Further still, the potential customer's user interface may include a “Send” button 310A designed to indicate to the scheduling system that the potential customer would like to find an available groomer on a particular day for a scheduled meeting.

FIG. 3B illustrates a groomer's user interface associated with operations outlined in FIG. 2, in accordance with one embodiment. As an option, the groomer's user interface may include a visual calendar 302B designed to allow selection of a potential day for a scheduled meeting. In addition, the groomer's user interface may include an advance button 306B and a back button 304B designed to move between subsequent and previous calendar months. Further, the groomer's user interface may include indication of the groomer's virus infection status certification 308B. Further still, the groomer's user interface may include a “Send” button 310B designed to indicate to the scheduling system that the groomer is available to meet with a potential client on a particular day for a scheduled meeting.

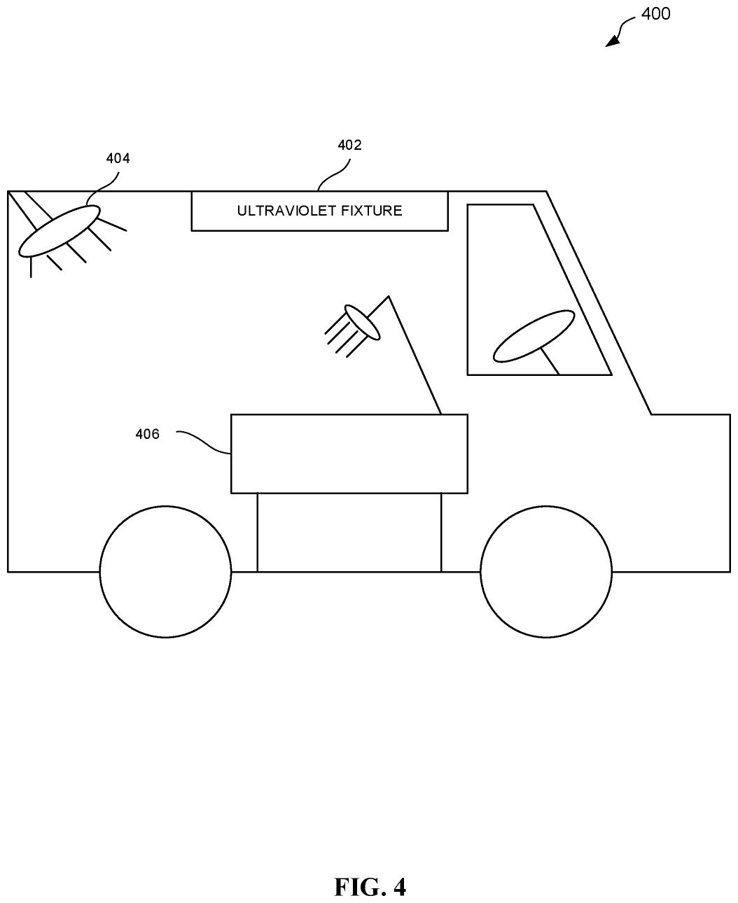

FIG. 4 illustrates an exemplary mobile pet grooming facility with a variety of disinfecting systems.

As shown, a mobile pet grooming facility 400 may include a variety of disinfecting systems. In one embodiment, the mobile pet grooming facility may include an ultraviolet light fixture 402 designed to eradicate viral specimens on exposed surfaces within the mobile pet grooming facility. In another embodiment, the mobile pet grooming facility may include a sanitizing vaporous spray apparatus 404 designed to sanitize the interior of the mobile pet grooming facility before and after a scheduled meeting. In yet another embodiment, the mobile pet grooming facility may include a pet bathing vessel 406 where sanitizer may be available with which to treat a pet's skin and fur during a scheduled pet grooming meeting.

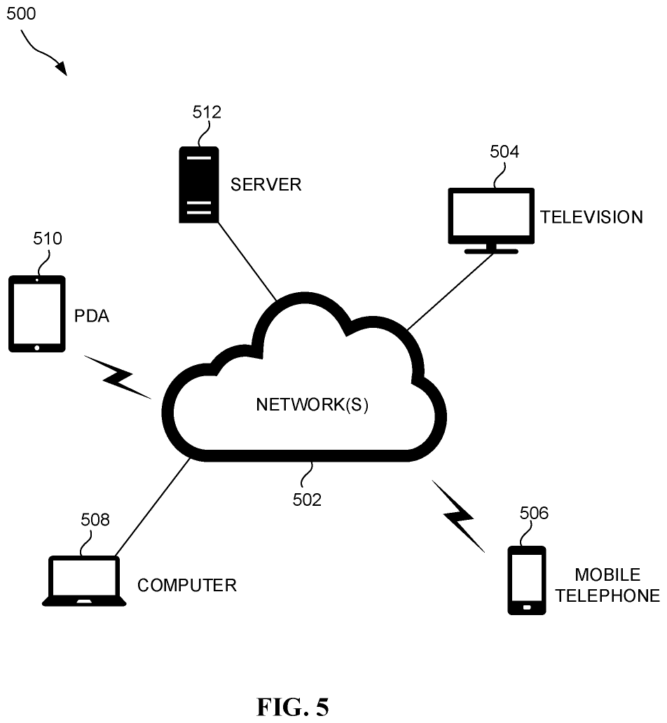

FIG. 5 illustrates a network architecture 500, in accordance with one possible embodiment. As shown, at least one network 502 is provided. In the context of the present network architecture 500, the network 502 may take any form including, but not limited to a telecommunications network, a local area network (LAN), a wireless network, a wide area network (WAN) such as the Internet, peer-to-peer network, cable network, etc. While only one network is shown, it should be understood that two or more similar or different networks 502 may be provided.

Coupled to the network 502 is a plurality of devices. For example, a server computer 512 and an end user computer 508 may be coupled to the network 502 for communication purposes. Such end user computer 508 may include a desktop computer, lap-top computer, and/or any other type of logic. Still yet, various other devices may be coupled to the network 502 including a personal digital assistant (PDA) device 510, a mobile phone device 506, a television 504, etc.

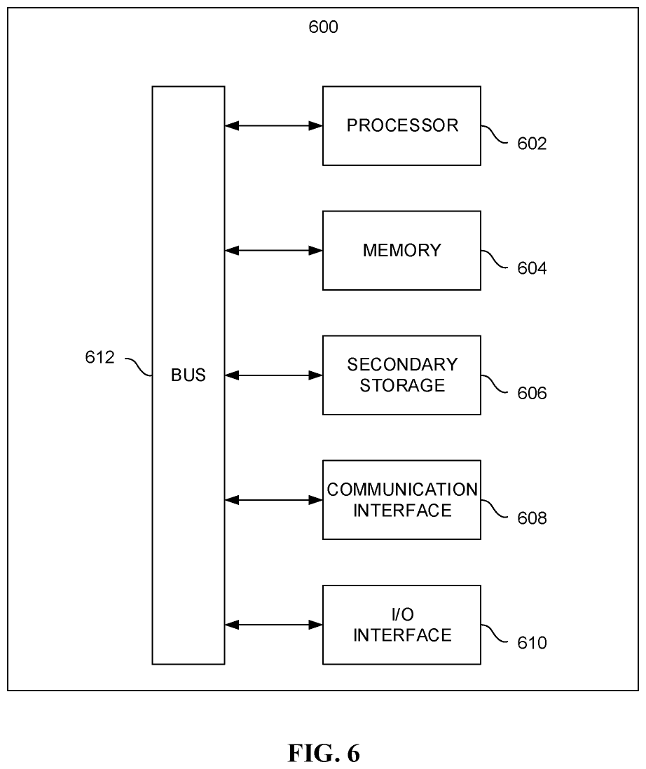

FIG. 6 illustrates an exemplary system 600, in accordance with one embodiment. As an option, the system 600 may be implemented in the context of any of the devices of the network architecture 500 of FIG. 5. Of course, the system 600 may be implemented in any desired environment.

As shown, a system 600 is provided including at least one central processor 602 which is connected to a communication bus 612. The system 600 also includes main memory 604 [e.g. random access memory (RAM), etc.]. The system 600 also includes a graphics processor 608 and a display 610.

The system 600 may also include a secondary storage 606. The secondary storage 606 includes, for example, a hard disk drive and/or a removable storage drive, representing a floppy disk drive, a magnetic tape drive, a compact disk drive, etc. The removable storage drive reads from and/or writes to a removable storage unit in a well-known manner.

Computer programs, or computer control logic algorithms, may be stored in the main memory 604, the secondary storage 606, and/or any other memory, for that matter. Such computer programs, when executed, enable the system 600 to perform various functions (as set forth above, for example). Memory 604, storage 606 and/or any other storage are possible examples of non-transitory computer-readable media. It is noted that the techniques described herein, in an aspect, are embodied in executable instructions stored in a computer readable medium for use by or in connection with an instruction execution machine, apparatus, or device, such as a computer-based or processor-containing machine, apparatus, or device. It will be appreciated by those skilled in the art that for some embodiments, other types of computer readable media are included which may store data that is accessible by a computer, such as magnetic cassettes, flash memory cards, digital video disks, Bernoulli cartridges, random access memory (RAM), read-only memory (ROM), and the like.

As used here, a “computer-readable medium” includes one or more of any suitable media for storing the executable instructions of a computer program such that the instruction execution machine, system, apparatus, or device may read (or fetch) the instructions from the computer readable medium and execute the instructions for carrying out the described methods. Suitable storage formats include one or more of an electronic, magnetic, optical, and electromagnetic format. A non-exhaustive list of conventional exemplary computer readable medium includes: a portable computer diskette; a RAM; a ROM; an erasable programmable read only memory (EPROM or flash memory); optical storage devices, including a portable compact disc (CD), a portable digital video disc (DVD), a high definition DVD (HD-DVD™), a BLU-RAY disc; and the like.

It should be understood that the arrangement of components illustrated in the Figures described are exemplary and that other arrangements are possible. It should also be understood that the various system components (and means) defined by the claims, described below, and illustrated in the various block diagrams represent logical components in some systems configured according to the subject matter disclosed herein.

For example, one or more of these system components (and means) may be realized, in whole or in part, by at least some of the components illustrated in the arrangements illustrated in the described Figures. In addition, while at least one of these components are implemented at least partially as an electronic hardware component, and therefore constitutes a machine, the other components may be implemented in software that when included in an execution environment constitutes a machine, hardware, or a combination of software and hardware.

More particularly, at least one component defined by the claims is implemented at least partially as an electronic hardware component, such as an instruction execution machine (e.g., a processor-based or processor-containing machine) and/or as specialized circuits or circuitry (e.g., discreet logic gates interconnected to perform a specialized function). Other components may be implemented in software, hardware, or a combination of software and hardware. Moreover, some or all of these other components may be combined, some may be omitted altogether, and additional components may be added while still achieving the functionality described herein. Thus, the subject matter described herein may be embodied in many different variations, and all such variations are contemplated to be within the scope of what is claimed.

In the description above, the subject matter is described with reference to acts and symbolic representations of operations that are performed by one or more devices, unless indicated otherwise. As such, it will be understood that such acts and operations, which are at times referred to as being computer-executed, include the manipulation by the processor of data in a structured form. This manipulation transforms the data or maintains it at locations in the memory system of the computer, which reconfigures or otherwise alters the operation of the device in a manner well understood by those skilled in the art. The data is maintained at physical locations of the memory as data structures that have particular properties defined by the format of the data. However, while the subject matter is being described in the foregoing context, it is not meant to be limiting as those of skill in the art will appreciate that various of the acts and operations described hereinafter may also be implemented in hardware.

To facilitate an understanding of the subject matter described herein, many aspects are described in terms of sequences of actions. At least one of these aspects defined by the claims is performed by an electronic hardware component. For example, it will be recognized that the various actions may be performed by specialized circuits or circuitry, by program instructions being executed by one or more processors, or by a combination of both. The description herein of any sequence of actions is not intended to imply that the specific order described for performing that sequence must be followed. All methods described herein may be performed in any suitable order unless otherwise indicated herein or otherwise clearly contradicted by context.

The use of the terms “a” and “an” and “the” and similar referents in the context of describing the subject matter (particularly in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. Furthermore, the foregoing description is for the purpose of illustration only, and not for the purpose of limitation, as the scope of protection sought is defined by the claims as set forth hereinafter together with any equivalents thereof entitled to. The use of any and all examples, or exemplary language (e.g., “such as”) provided herein, is intended merely to better illustrate the subject matter and does not pose a limitation on the scope of the subject matter unless otherwise claimed. The use of the term “based on” and other like phrases indicating a condition for bringing about a result, both in the claims and in the written description, is not intended to foreclose any other conditions that bring about that result. No language in the specification should be construed as indicating any non-claimed element as essential to the practice of the invention as claimed.

The embodiments described herein included the one or more modes known to the inventor for carrying out the claimed subject matter. Of course, variations of those embodiments will become apparent to those of ordinary skill in the art upon reading the foregoing description. The inventor expects skilled artisans to employ such variations as appropriate, and the inventor intends for the claimed subject matter to be practiced otherwise than as specifically described herein. Accordingly, this claimed subject matter includes all modifications and equivalents of the subject matter recited in the claims appended hereto as permitted by applicable law. Moreover, any combination of the above-described elements in all possible variations thereof is encompassed unless otherwise indicated herein or otherwise clearly contradicted by context.

What is claimed is:

1. A method, comprising:
receiving first information regarding a virus infection status of a first person;
receiving second information regarding a virus infection status of a second person;
based on the first information and the second information, conditionally scheduling a meeting between the first person and the second person.
